(19) United States
(12) Patent Application Publication
(10) Pub. No.: US 2003/0143282 A1
(43) Pub. Date: Jul. 31, 2003

(54) Adenosine A3 receptor agonist

(76) Inventor: Pnina Fishman, Herzliya (IL)

Correspondence Address: BIRCH STEWART KOLASCH & BIRCH, PO BOX 747, FALLS CHURCH, VA, 22040-0747, US

(21) Appl. No.: 10/056,484

(22) Filed: Jan. 28, 2002

(51) Int. Cl. A61K 35/14; A61K 35/34

(52) U.S. Cl. 424/548; 424/529

(57) ABSTRACT

The present invention relates to a naturally occurring low molecular weight adenosine A3 receptor agonist (LMW-A3RAg) which is preferably obtained from a vertebrate tissue or a vertebrate-derived cell by extraction in a liquid medium. The LMW-A3RAg of the invention is characterized by the following feature: (i) it is obtainable from animal-derived tissue or cells; (ii) it filters through a filter with a maximal molecular weight cut-off of about 3,000 Daltons; (iii) it is water soluble, heat stable, non-proteinaceous and resistant to adenosine deaminase activity.

The invention also concerns pharmaceutical compositions comprising the naturally occurring LMW-A3RAg of the invention and therapeutic methods comprising administering to a subject in need an effective amount of the naturally occurring A3RAg for achieving a therapeutic effect, the therapeutic effect comprises inhibition of adenylate cyclase in target cells.

### FIELD OF THE INVENTION

The present invention is generally in the field of medicine and concerns agents, which selectively affect growth and proliferation of cells. The present invention also concerns the use of such agents in prevention or therapy of malignant disorders and diseases, as well as in chemoprotection during chemotherapeutic treatments.

### PRIOR ART

The following is a list of prior art which is considered to be pertinent for describing the state of the art in the field of the invention. Acknowledgement of these references herein will be made by indicating the number from their list below within brackets.

1. Sarma D P, Weilbaccher T G, and Love G L (1985) Intramyofiber metastasis in skeletal muscle. J. Surg, Oncol. 30, 103-105.

2. Pellegrini A E (1979) Carcinoma of the lung occurring as a skeletal muscle mass. Arch. Surg. 114, 550-555.

3. Merimsky O, Levin T, and Chaitchik S (1990) Recurrent solitary metastasis of renal cell carcinoma in skeletal muscles. Tumori. 76, 407-409.

4. Djaldetti M, Sredni B, Zigelman R, Verber M, and Fishman P (1996) Muscle cells produce a low molecular weight factor with anti-cancer activity. Clin. Exp. Matastasis. 14, 189-96.

5. Bar-Yehuda S, Farbstein T, Barer F, Ohana G and Fishman P (1999) Oral administration of muscle derived small molecules inhibits tumor spread while promoting normal cell growth in mice. Clin. Exp. Metastasis. 17, 531-535.

6. Fishman P, Bar-Yehuda S, and Vagman L (1998) Adenosine and other low molecular weight factors released by muscle cells inhibit tumor cell growth. Cancer Res. 58, 3181-3187.

7. Linden J (1991) Structure and function of A1 adenosine receptors. The FASEB J 5, 2668-2676.

8. Stiles G (1990) Adenosine receptors and beyond: molecular mechanisms of physiological regulation. Clin. Res. 38, 10-18.

9. Fishman P, Bar-Yehuda S, Farbstein T, Barer F, and Ohana (2000). Adenosine Acts as a Chemoprotective Agent By Stimulating G-CSF Production: A Role for A1&A3 Adenosine Receptors. J. Cellular Physiol, 183, 393-398.

10. Fishman P, Bar-Yehuda S, Ohana G, Pathak S, Wasserman L, Multani A S, and Barer F (2000) Adenosine Arts as an Inhibitor of Lymphoma Cell Growth: a Major Role for the A3 Adenosine Receptor. European J. Cancer 36,1452-1458.

11. Lasser A, and Zacks I S (1982) Intraskeletal myofiber metastasis of breast carcinoma. Hum. Pathol. 13, 1045-1046.

12. Slatkin D N, and Pearson J (1976) Intramyofiber metastases in skeletal muscle. Hum. Pathol. 7, 347-349.

13. Ioachim H L (1983) Tumor cells within skeletal muscle cells. Hun. Pathol. 14, 924-929.

14. U.S. Pat. No. 5,962,331

15. WO01/07060

16. WO 01/19360

### BACKGROUND OF THE INVENTION

The resistance of muscle tissue to the development of tumor metastases is a well-recognized clinical phenomenon (1-3). It was also reported that small molecules (<800 dalton) present in muscle cell conditioned medium (MCM) exerted an inhibitory effect on the growth of various tumor cell lines and simultaneously stimulate the proliferation of normal bone marrow cells (4). These small molecules in the MCM were found to be water soluble, heat stable and resistant to the activity of proteolytic enzymes. When administered orally to mice, it inhibited the development of melanoma and sarcoma lung metastases, while protecting against the myelotoxic effects of chemotherapy (5).

Recently, a component in the MCM, that exerts the anti-proliferative effect was identified as adenosine, which showed to exert a differential effect on tumor and normal cell growth in vitro (6). Adenosine, a ubiquitous nueleoside, is released into the extracellular environment from metabolically active or stressed cells. It is known to act as an important regulatory molecule by binding to specific G-protein associated A1, A2a, A2b and A3 cell surface receptors (7,8). Using antagonists to the adenosine receptors, it was revealed that adenosine exerted its in vitro inhibitory effect as well as its stimulatory activity through the activation of the A3 adenosine receptor (A3AR)(9,10).

### SUMMARY OF THE INVENTION

The present invention is based on the surprising finding that MCM contains molecules, which are natural agonists to the A3AR. It was hitherto unknown that natural molecules that are agonists to the A3AR exist.

Accordingly, the invention provides, by one of its aspects, an endogenous, low molecular weight adenosine A3 receptor agonist (LMW-A3RAg).

The term “low molecular weight (LMW)” used herein refers to a molecular weigh, as determined by ultrafiltration, which is less than about 3,000 Daltons and particularly less than about 1,000 Daltons. It should be clear to the artisan that these molecular weights are approximations and cannot be regarded as exact figures.

The term “agonist” used herein refers to any compound, which is capable of inhibiting (suppressing/reducing) adenylate cyclase. The naturally occurring adenosine A3 receptor agonist according to the invention may be either a full agonist or a partial agonist of the adenosine A3 receptor. As used herein, a compound is a “full agonist” of an adenosie A3 receptor if it is able to fully inhibit adenylate cyclase activity, while a compound is a “partial agonist” of an adenosine A3 receptor if it is able to partially inhibit adenylate cyclase activity.

The term “naturally occurring A3RAg” which is used herein interchangeably with the term “naturally occurring low molecular weight A3RAg”, “endogenous LMW-A3RAg” or interchangeably with the more general term “active agent” refers to an agent secreted by or shed from a cell within a living body which has an A3AR agonist activity. The agent may be a single molecule, a group of molecules operating together in an additive or synergistic manner or a molecular complex having an A3AR agonist activity. The A3AR agonist activity is manifested in binding to A3AR. This can be determined by one of a variety of binding or displacement assays. For example, the ability of an agent to bind to A3AR can be demonstrated by the use of membranes containing A3ARs bound to a radio labeled synthetic agonists. The existence of an agent that is an agonist to the A3AR agonist will cause the dissociation of the radio-labeled agonist from the membranes and reduction in radioactivity that remains bound to the membranes and an increase in radioactivity released into the surrounding medium. The A3AR agonist activity can also be manifested through its biological activity that includes ability to inhibit proliferation of tumor cells both in vitro and in vivo (the latter can be manifested through both oral or parenteral administration); ability to induce proliferation of a variety of non-tumor cells such as bone marrow cells, fibroblasts or muscle cells of a constitutive muscle cell line in vitro; ability to induce white-blood cells and neutrophils proliferation as well as induce production of G-CSF (granulocyte colony stimulating factor) in vivo following parenteral or oral administration; and in general, the ability to induce any of the A3AR activities disclosed in WO 01/19360 (16).

By a second of its aspects, the present invention provides a pharmaceutical composition comprising, as an active ingredient a therapeutically effective amount of a naturally occurring LMW-A3RAg and a pharmaceutically acceptable excipient.

The term “effective amount” used herein refers to an amount determined by such considerations as may be known in the art. The amount must be effective to achieve the desired therapeutic effect. The therapeutic effect may be manifested in the reduction or suppression of adenylate cyclase activity in the target cells or tissue. The effective amount in such a case is an amount effective to suppress the activity of adenylate cyclase in the target cells or tissue. The therapeutic effect may be manifested also in reduction in the rate of growth and proliferation of tumor cells as can be gauged, for example, through measuring of tumor size, determining the number or rate of tumor metastasis. An effective amount in such latter case is thus an amount effective to obtain such tumor growth reduction. The therapeutic effect may further be manifested in a myelostimulatory effect, namely in stimulation of proliferation of myeloid cells, in particular neutrophils, e.g. in order to counter drug-induced myelotoxicity, e.g. such caused by chemotherapeutic drugs. In this latter case an effective amount is an amount that is effective in inducing proliferation of myeloid cells. The effective amount in this latter case is thus an amount effective to stimulate proliferation of the myeloid cells. As can readily be appreciated by the artisan, the effective amount depending, inter alia, on the type and severity of the disease to be treated, the treatment regime, at times also on the age of the treated individual or its gender, etc. Determination of the effective amount is within reach of the artisan.

The term “pharmaceutically acceptable excipient” as used herein refers to any substance combined with the active agent and include, without being limited thereto, diluents, additives, carriers, solid or liquid fillers or encapsulating materials which are typically added to formulations to give them a form or consistency when it is given in a specific form, e.g. in pill form, as a simple syrup, aromatic powder, and other various elixirs. The additives may also be substances for providing the formulation with stability, sterility and isotonicity (e.g. antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor et. The pharmaceutically acceptable excipients can be any of those conventionally used and is limited only by chemico-physical considerations, such as solubility and lack of reactivity with the compound, and by the route of administration.

Preferably, the pharmaceutically acceptable excipients are inert, non-toxic materials, which do not react with the active ingredient of the invention. Yet, the excipient may be designed to enhance the binding of the active agent to its receptor. Further, the term additive may also include adjuvants, which are substances affecting the action of the active ingredient in a predictable way.

By a third of its aspects, the present invention provides a method for a therapeutic treatment comprising administering to a subject in need an effective amount of at least one endogenous A3RAg for achieving a therapeutic effect, the therapeutic effect comprises inhibition of adenylate cyclase in target cells.

The term “treatment” as used herein refers to the administering of a therapeutic effective amount of the naturally occurring A3RAg provided by the present invention, the amount being sufficient to achieve a therapeutic effect leading to amelioration of undesired symptoms associated with a certain disease, disorder or condition, prevention of the manifestation of such symptoms before they occur, slowing down the deterioration of the symptoms, slowing down the progression of the disease or disorder, lessening the severity or curing the disease, improving of the survival rate or achieving more rapid recovery of a subject suffering from the disease, prevention of the disease from occurring or a combination of two or more of the above.

The term “target cells” as used herein refers to cells that have A3AR on their membrane and which are associated with the manifestation of a disease, disorder or condition.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 is a bar graph showing the effect of adenosine, muscle cell cultured medium (MCM) and the combination of muscle cell cultured medium and adenosine deaminase (MCM+ADA) on the development of lung metastases in mice inoculated with B16-F10 melanoma cells.

FIGS. 2A-2B are bar graphs showing the in vivo effect of adenosine, MCM and MCM+ADA on the number of white blood cells (WBC) (FIG. 2A) and percentage of neutrophils (FIG. 2B) in mice treated with 50 mg/kg body weight of cyclophosphamide.

FIGS. 3A-3B are bar graphs showing the effect of MCM, MCM+ADA and MCM+ADA+MRS 1220 (adenosine A3 receptor antagonist) on the growth of B16-F10 melanoma cells (FIG. 3A) and bone marrow cells (FIG. 3B) as measured by [³H]thymidine incorporation assay.

### DETAILED DESCRIPTION OF THE INVENTION

The resistance of muscle tissue to the development of metastases has been previously investigated (11-13, 14). In addition, it was reported that low molecular weight fraction of MCM exhibited a unique characteristic of differentiating between tumor and normal cells. It inhibited tumor cell growth and maintained bone marrow cell proliferation in vitro (4). Similar data was reproduced in vivo when MCM was orally administered to mice with melanoma or sarcoma. It suppressed tumor development and induced a prolongation of survival time in the treated mice. Moreover, it acted as a chemoprotective agent by preventing the myelotoxic effects of cyclophosphamide (5). At a later stage, adenosine was considered as one of the MCM components, which exhibited in vitro, a differential effect on tumor and normal cell growth (6).

Surprisingly it has now been found that adenosine is not responsible for the in vivo efficacy of the MCM. As will be shown in the following specific examples, this finding is based on the fact that adenosine failed to exert anti-cancer or chemoprotective effects when given in vivo orally or intraperitoneally (unlike the MCM). Moreover, when adenosine was eliminated from the MCM by the use of adenosine deaminase (MCM treated by adenosine deaminase is referred to herein as “MCM+ADA”), the adenosine-free MCM still retained its dual effect in vitro and in vivo (namely inhibition of proliferation of tumor cells and stimulation of proliferation of normal cells, notably myeloid cells and particularly neutrophils. For example, in vitro, MCM+ADA inhibited the proliferation of melanoma cells and induced proliferation of bone marrow cells. As also shown hereinbelow, these activities were mediated through the A3 adenosine receptor since the MRS-1220 A3 antagonist blocked this dual effect. Thus, it was concluded that the active ingredient in MCM+ADA includes an endogenous adenosine A3 receptor agonist.

Therefore, the present invention provides, by a first of its aspects one or more natural agonists to the A3 adenosine receptor, referred to herein by the term “endogenous low molecular weight adenosine A3 receptor agonist” (LMW-A3RAg). The high therapeutic efficacy of synthetic agonists to A3AR and the biological effect of the MCM+ADA suggests that LMW-A3Rag of the invention has a highly promising therapeutic potential.

The LMW-A3Rag of the invention is obtainable from a vertebrate tissue or a vertebrate-derived cell or by extraction in a liquid medium. According to one non-limiting embodiment, the animal tissue from which the LMW-A3RAg may be obtained, is a muscle tissue. The muscle tissue may be a bovine muscle tissue, pork muscle tissue, fowl muscle tissue and others. The muscle tissue may be in the form of fresh, preserved or frozen tissue. The LMW-A3RAg of the invention is also obtainable from a medium conditioned by vertebrate source cells, e.g. cells of mammalian or animal origin. According to one embodiment, the source cell (the cell which secrete or shed the naturally occurring LMW-A3RAg) are muscle cells, while according to another embodiment, the source cells are white blood cells.

The LMW-A3RAg of the invention may be obtained from other source cells. The present invention is, however, not limited to LMW-A3RAg derived from muscle or white blood cell. On the contrary, equipped with the knowledge gained by the findings in accordance with the invention and by employing standard skills and knowledge available, the artisan will have no difficulties in finding other sources of LMW-A3RAg giving rise to other LMW-A3RAgs, which fall within the scope of the present invention.

The LMW-A3RAg of the invention can be characterized by the following features: it has a molecular weight of less than about 3,000 Dalton (as it filters through a filter with a maximal molecular weight cut-off of about 3,000 Daltons); it is water soluble; heat-stable; non-proteinaceous; and as surprisingly found herein, it is resistant to degradation by adenosine deaminase (ADA).

The enzyme adenosine deaminase metabolizes adenosine. The metabolization of adenosine to inosine by the enzyme is shown in the following

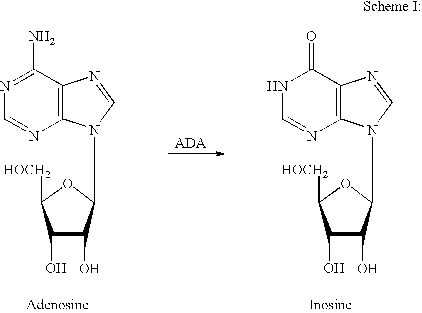

The naturally occurring Scheme I LMW-A3RAg may be obtained by several methods. For example, the LMW-A3RAg are obtained from an animal derived tissue, by treating the tissue, e.g. muscle tissue derived from chicken, in a liquid medium under conditions in which a composition of matter contained within the tissue is freed into the medium as a supernatant. The supernatant is then separated from the cell matter and filtered such that a fraction comprising substances having a molecular weigh of below about 3,000 Daltons is collected.

Alternatively, the LMW-3RAg of the invention may be obtained from a cell cultured medium, e.g. muscle cells or white blood cells. Accordingly cells are grown in a growth medium under conditions in which the cells produce, secrete or shed into their surrounding medium at least one LMW-A3RAg; the cells are then separated from the medium to obtain a supernatant which is collected and subjected to ultrafiltration through a membrane with a molecular cut-off of 3,000 Daltons.

The low molecular weigh fraction containing the A3RAg may be treated to remove therefrom the naturally occurring adenosine, e.g. by treatment of the fraction with adenosine deaminase. Further, it may be maintained in its original liquid form or dried, e.g. by lyophilization. Yet further, the LMW-A3RAg containing fraction may be in the form of a purified or substantially purified LMW-A3RAg. In order to obtain a more purified active agent, the fraction obtained after filtration (which may or may not have been treated also with adenosine deaminase) is subjected to additional purification steps, such as, for example, fractionation by chromatography, typically high pressure liquid chromatography (HPLC), e.g. reverse phase HPLC or size exclusion chromatography; additional filtration stages; purification by dialysis, etc., as long as the selected fraction maintains an adenosine A3 receptor agonist activity.

The present invention also provides a pharmaceutical composition comprising as an active ingredient a therapeutically effective amount of at least one naturally occurring LMW-A3RAg and a pharmaceutically acceptable excipient.

The LMW-A3RAg of the invention employed may be an isolated agonist or a synthetic agonist having the same chemical structure of the naturally occurring LMW-A3RAg.

The LMW-A3RAg of the invention may be formulated for administration via any medically acceptable means. Suitable means of administration include inter alia, oral, rectal, topical or parenteral (including subcutaneous, intramuscular, and intravenous) administrations. According to one preferred embodiment, the pharmaceutical composition comprising the LMW-A3RAg of the invention is formulated for oral administration. Such formulations, include, inter alia, tablets, suspensions, solutions, emulsions, capsules, powders, syrups and the like are usable.

To this end, the composition of the invention may contain excipients for facilitating oral delivery of the active agent. Formulations suitable for oral administration can consist of (a) liquid solutions in which the active agent is dissolved in diluents, such as water, saline, or orange juice; (b) capsules, sachets, tablets, lozenges, and troches, each containing a predetermined amount of the active agent, as solids or granules; (c) powders; (d) suspensions in an appropriate liquid; and (e) suitable emulsions.

Liquid formulations may include diluents, such as water and alcohols, for example, ethanol, benzyl alcohol and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose acacia, gelatin, guar gum, colloidal silicon dioxide, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active agent in a flavor, usually sucrose and acacia or tragacanth, as well as pastilles comprising the active ingredient in an inert base, such as gelatin and glycerin, or sucrose and acacia, emulsions, gels, and the like. Non-aqueous vehicles such as cottonseed oil, sesame oil, olive oil, soybean oil, corn oil, sunflower oil, or peanut oil and ester, such as isopropyl myristate, may also be used as solvent systems for the composition of the present invention.

Alternatively, the composition of the invention may be formulated for parenteral administration. To this end, the compositions will generally be formulated in a unit dosage injectable form (solution, suspension, emulsion). Pharmaceutical formulation suitable for injection may include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, lipid polyethylene glycol and the like), suitable mixtures thereof and vegetable oils.

Suitable fatty acids for use in parenteral formulations include oleic acid, stearic acid, and isostearic acid. Ethyl oleate and isopropyl myrisate are examples of suitable fatty acid esters.

Suitable soaps for use in parenteral formulations include fatty alkali metal, ammonium and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxy-ethylenepolypropylene copolymers, (d) amphoteric detergents such as, for example, alkyl-β-aminopriopionates, and 2-alkyl-imidazoline quaternary ammonium salts, and (3) mixtures thereof.

Further, in order to minimize or eliminate irritation at the site of injection, the compositions may contain one or more nonionic surfactants having a hydrophile-lipophile balance (HLB) of from about 12 to about 17. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

The amount of the LMW-A3RAg of the invention required to be effective as agonist or partially agonist of the adenosine receptor, will, of course, vary with the individual mammal being treated and is ultimately at the discretion of the medical or veterinary practitioner. The factors to be considered include the condition being treated, the route of administration, the nature of the formulation, the mammal's body weight surface area, age and general conditions.

The total daily dose of the active agent may be formulated as a single dose, multiple dose, e.g. for administration two to six times per day, or by intravenous infusion for a selected duration.

The invention further provides a method for a therapeutic treatment comprising administering to a subject in need an effective amount of at least one naturally occurring A3RAg for achieving a therapeutic effect, the therapeutic effect comprises inhibition of adenylate cyclase in cells.

The naturally occurring A3RAg may be administered alone or in combination with an additional therapeutic treatment. The additional therapeutic agent may be provided to the subject in need together with the natural A3RAg (e.g. formulated in the same composition, or by concomitant administration of two separate compositions), or with a time interval suitable for achieving the desired therapeutic effect.

The LMW-A3RAg of the present invention may be effective for the treatment of numerous diseases and disorders which require for their treatment reduction (fully or partially) of the adenylate cyclase activity in the diseased cells. Examples for diseases and disorders against which A3RAg are known to be effective, include, without being limited thereto, cancer and viral diseases.

The LMW-A3RAg of the invention may also be employed as a chemoprotective agent. Further, cytoprotective effect towards cardiac myocytes or brain cells have previously been attributed to adenosine A3R agonists.

Obviously, many modifications and variations of the present invention are possible in light of the above teaching. Accordingly, it should be understood that any other use of the endogenous LMW-A3RAgs or their synthetic counterparts which is within the scope of the appended claims forms part of the present invention and that the invention may be practiced otherwise than as specifically described hereinafter

### SPECIFIC EXAMPLES

Materials and Methods

Preparation of MCM

Muscle conditioned medium (MCM) was obtained from the L-8 cell line (consisting of proliferating myoblasts) purchased from the American Type Tissue Culture Collection, Rockville, Md. (ATCC). The cells were routinely maintained in DMEM containing 4.5 gr % glucose and 15% Fetal Bovine Serum (FBS) (Biological Industries, Beit Haemek, Israel).

To prepare MCM, cultures were grown until confluence, medium discarded, cells washed twice with phosphate buffered saline (PBS) and then incubated for an additional 20 hours in PBS. At the end of the incubation period, the supernatant was collected, centrifuged and filtered through 0.22 μm filter. The MCM was subjected to ultrafiltration through an Amicon membrane with a molecular cut-off of 3 kD.

Tumor and Normal Cells

The B-16-F10 murine melanoma cell line was used in the in vitro and in vivo experiments. Cells were maintained in RPMI medium containing 10% FBS, penicillin and streptomycin. They were transferred twice weekly to a freshly prepared medium.

Bone marrow cells were obtained from the femur of C57BL/6J mice. Cells were disaggregated by passing through a 25 G needle.

In addition the HCT-116 human colon carcinoma tumor cell line was used in in vivo studies.

Cell Proliferation Assays

[³H]-tymidine incorporation assay was used to evaluate cell growth. 1.5×10⁴/ml B-16-F10 melanoma or 3×10⁵/ml bone marrow cells were cultured in RPMI medium containing 10% FBS in 96 well microtiter plates. These cultures were incubated in the presence of MCM in a concentration of 50%. Since MCM was prepared in PBS, cultures containing tumor or bone marrow cells suspended in 50% PBS served as controls. During the last 18 hours of incubation, each well was pulsed with 1 μCi [³H]-thymnidine. The cells were harvested and the [³H]-thymnidine uptake was determined in an LKB liquid scintillation counter (LKB, Piscataway, N.J., USA).

Results were expressed as % of cell proliferation inhibition or stimulation, calculated according to the following formula:

$$\begin{matrix}\% inhibition = 100 - \frac{A \times 100}{B} \\ \% stimulation = \frac{A \times 100}{B} - 100\end{matrix}$$

where A represents the cell count of sample and B represents the cell count of the control. According to this calculation control values are 0% of inhibition or stimulation.

One activity unit was defined as the amount of MCM exerting 50% proliferation inhibition of the B-16-F10 melanoma cells.

Elimination of Adenosine From MCM by Treatment With Adenosine Deaminase (ADA)

To eliminate adenosine from MCM preparations, adenosine deaminase (ADA) (Sigma, Chemical Co. St. Louis, Mo., USA) was added to the MCM for 1 hour. To remove the enzyme, the preparation was ultrafiltrated through a 3000 Dalton Amicon membrane. This sample was designated as MCM+ADA and its effect on the proliferation of tumor or bone marrow cells was examined as described above.

Effect of A3AR Antagonist on the Activity of MCM+ADA

In this set of experiments, the question whether the effect of MCM+ADA on tumor or normal cells was mediated through A3AR was examined. To this end, 1.5×10⁴/ml B-16-F10 melanoma or 3×10⁵/ml bone marrow cells were cultured in RPMI medium containing 10% PBS in 96 well microtiter plates. These cultures were pre-incubated for 30 min. in the presence of 0.1, 0.05 and 0.001 μM of the A3AR antagonist 9-chloro-2-(2-furanyl)-5-[(phenylacetyl)amino] [1,2,4,]-triazolo[1,5-c] quinazoline (MRS-1220)(RBI Massachusetts, USA). At the end of this incubation period, MCM+ADA, at a final concentration of 50%, was added to the cultures for additional 48 h. As controls, cells were pre-incubated with MRS-1220 for 30 min and then with 50% PBS+ADA for 48 h. During the last 18 hours of incubation, each well was pulsed with 1 μCi [³H]-thymidine. [³H]-thymidine uptake was determined as described above.

Effect of Synthetic A3AR Agonist on the Proliferation of Tumor and Bone Marrow Cells

A synthetic agonist to the A3AR, 1-Deoxy-1-[6-[[(3-iodophenyl)methyl]amino]-9H-purine-9-yl]-N-methyl-β-D-ribofuranuronamide (IB-MECA) (Sigma, Chemical Co. St. Louis, Mo., USA), was used to examine its effect on B16-F10 melanoma and murine bone marrow cells. A stock solution was prepared by dissolving 5 mg IB-MECA in 1 ml DMSO. Further dilutions were performed in RPMI for in vitro studies and PBS for in vivo experiments. Cells, either 1.5×10⁴/ml B-16-F10 melanoma or 3×10⁵/ml bone marrow cells were cultured in RPMI medium containing 10% PBS in 96 well microtiter plates. IB-MECA at concentrations of 0.1, 0.01 and 0.001 μM was added to these cell cultures for 48 h. [³H]-thymidine uptake was determined as described above.

In Vivo Studies

Experiments were performed in accordance with the guidelines established by the Institutional Animal Care and Use Committee at the Rabin Medical Center, Petah Tikva, Israel.

To examine the effect of the different preparations on tumor cell growth, B16-F10 (2.5×10⁵) melanoma cells were intravenously inoculated to C57BL/6J mice. Each group contained 20 mice and experiments were repeated at least 4 times. Mice were treated orally according to the following protocol starting one day after tumor inoculation:

1. Adenosine—268 μg/kg body weight

2. MCM 4AU twice daily.

3. MCM+ADA, 4AU twice daily.

4. Vehicle—twice daily.

An additional group of mice was treated daily intraperitoneally with adenosine (268 μg/kg body weight).

Mice were sacrificed after fifteen days, lungs removed and black metastatic foci were counted using a Dissecting Microscope.

Tumor growth was evaluated every 4 days, starting 12 days following tumor inoculation, by measuring width (W) and length (L). Tumor size was calculated according to the following formula:

$$Tumor Size = \frac{{( W )}^{2} \times L}{2}$$

The myeloprotective effect of the various preparations were examined by injecting mice intraperitoneally with 50 mg/kg body weight of cyclophosphamide. Each group contained 10 mice and experiments were repeated at least 4 times. Adenosine, MCM (4 activity units) or MCM+ADA (4 activity units) were each orally administered 48 h and 72 h following the chemotherapy. After 120 h blood samples were withdrawn. The number of leukocytes and percent of neutrophils were evaluated.

Statistical Analysis

The efficacy of the various agents in vitro and in vivo was evaluated using the student's t-test. The criterion for statistical significance was p<0.05.

Results

Adenosine is Not Responsible for the in vivo Activity of MCM

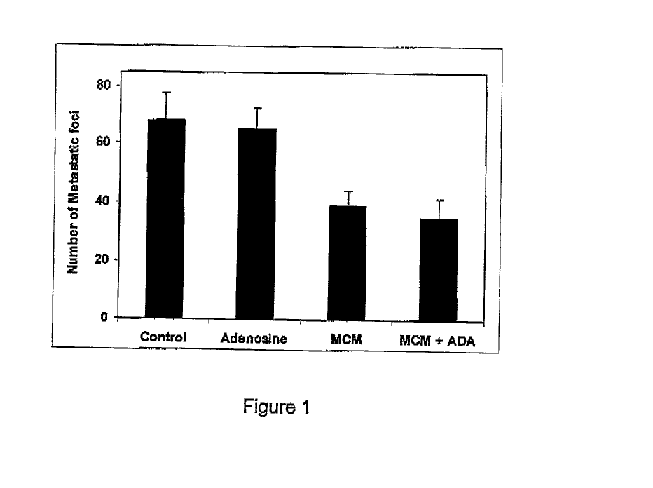

MCM or MCM+ADA were tested for their efficacy as inhibitors of melanoma lung metastatic foci growth in mice. All the preparations were administered to the mice via an oral route. MCM and MCM+ADA induced an inhibitory effect on tumor growth (42.7%±5.8, p<0.001; 49%±3.7, p<0.001, respectively, FIG. 1).

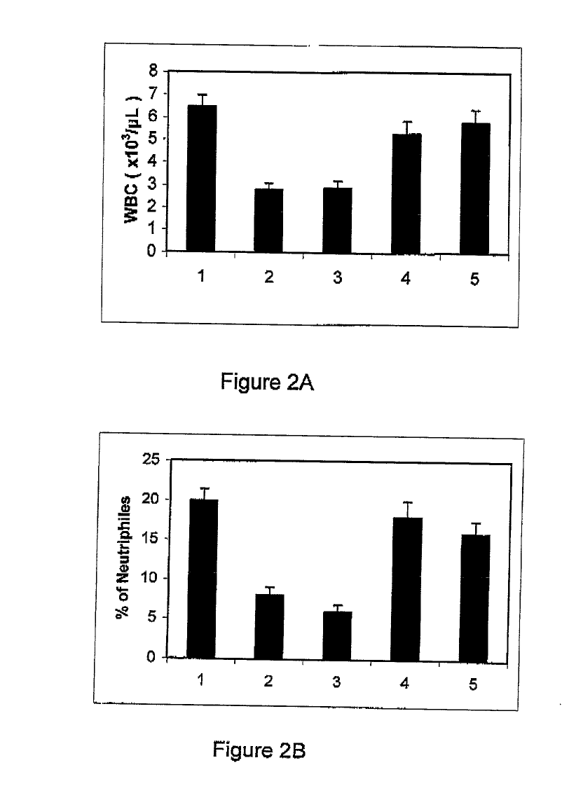

Administration of MCM or MCM+ADA to mice following chemotherapy, prevented the myelotoxic effects of the cytotoxic drug, i.e. induced an increase in the number of leukocytes (FIG. 2A) and percentage of neurtophils (FIG. 2B). Adenosine failed to inhibit tumor growth or act as a chemoprotective agent when administered orally or intraperitoneally.

Inhibition of Tumor Cell Growth and Stimulation of Bone Marrow Cell Proliferation is Mediated Through the A3 Adenosine Receptor

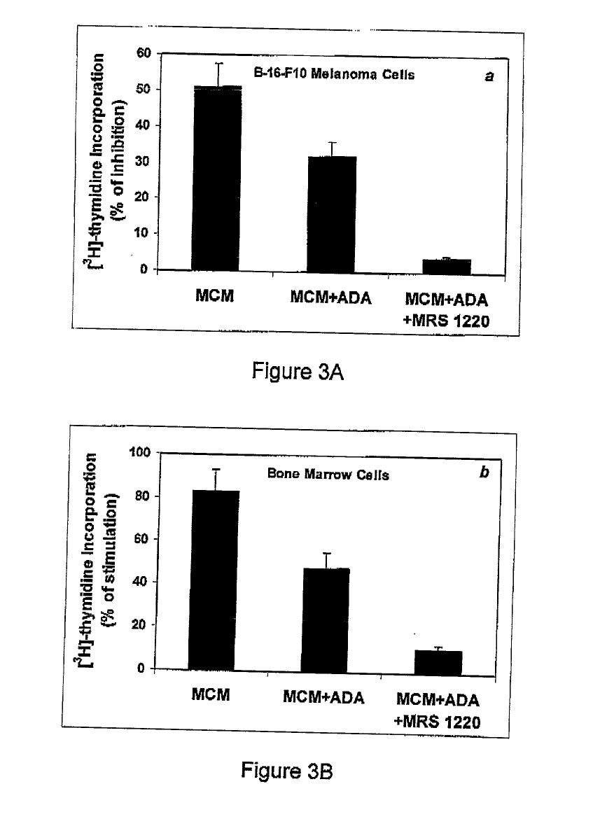

The above results demonstrate that the active component in MCM responsible for the dual activity in vivo, is not adenosine. However, since the MCM+ADA preparation still retained this same dual activity, it was presumed that both effects were mediated through the A3 adenosine receptor. To explore this assumption, B-16-F10 melanoma cells or murine bone marrow cells were incubated with MCM+ADA in the presence or absence of an antagonist to the A3 adenosine receptor. A statistically significant inhibition of [³H]-thymidine uptake following incubation with the MCM or MCM+ADA was observed. Since adenosine was removed from MCM+ADA, this preparation induced a decreased inhibitory effect. However, incubation of cells with MCM+ADA in the presence of MRS-1220, canceled most of the inhibitory effect (FIG. 3A).

Similarly, MCM and to a lesser extent MCM+ADA, stimulated the proliferation of bone marrow cells in vitro (83%±9.2 and 48%±7.1, respectively). Most of the stimulatory effect was lost following the incubation of bone marrow cells with MCM+ADA, in the presence of MRS-1220 (FIG. 3B). MRS-1220 by itself, had no effect on the proliferation of the B16-F10 or bone marrow cells. There was no difference between the results obtained with the various concentrations of the antagonist. Therefore, the results presented in FIGS. 3a and 3b represent results using a concentration of 0.001 μM of MRS-1220.

These results demonstrated that the inhibitory and the stimulatory activity of MCM were mediated through A3AR, the active component being an agonist to this receptor.

1. A naturally occurring low molecular weight adenosine A3 receptor agonist (LMW-A3RAg).

2. The LMW-A3RAg of claim 1, obtainable from a vertebrate tissue or a vertebrate-derived cell by extraction in a liquid medium.

3. The LMW-A3RAg of claim 2, obtainable from muscle tissue.

4. The LMW-A3RAg of claim 1, obtainable from medium conditioned by vertebrate source cells.

5. The LMW-A3RAg of claim 4, wherein said source cells are muscle cells.

6. The LMW-A3RAg of claim 4, wherein said source cells are white blood cells.

7. The LMW-A3RAg of claim 1, which is resistant to degradation by adenosine deaminase.

8. The LMW-A3RAg of claim 1, having the following characteristics;
(i) it is obtainable from animal-derived tissue or cells;
(ii) it filters through a filter with a maximal molecular weight cut-off of about 3,000 Daltons;
(iii) it is water soluble, heat stable, non-proteinaceous and resistant to adenosine deaminase activity.

9. A synthetic molecule having the same chemical structure as the agonist of claim 1.

10. A pharmaceutical composition comprising as an active ingredient, a therapeutically effective amount of at least one naturally occurring LMW-A3RAg and a pharmaceutically acceptable excipient.

11. A pharmaceutical composition comprising, as an active ingredient, a therapeutically effective amount of the molecule of claim 9.

12. The pharmaceutical composition of claim 10 or 11, formulated in any form suitable for oral administration.

13. A method for a therapeutic treatment comprising administering to a subject in need an effective amount of a naturally occurring A3RAg for achieving a therapeutic effect, the therapeutic effect comprises inhibition of adenylate cyclase in target cells.

14. The method of claim 13, wherein said LMW-A3RAg is administered in combination with an additional therapeutic treatment.

15. The method of claim 13 or 14, wherein said LMW-A3RAg is administered orally to the subject in need.

16. A method for a therapeutic treatment comprising administering to a subject in need an effective amount of a molecule according to claim 9.
